(19) United States
(12) Plant Patent Application Publication
(10) Pub. No.: US 2011/0126330 P1
(43) Pub. Date: May 26, 2011

(54) Apple tree named 'Gradiyel'

(50) ;

(76) Inventors: Alexandre Grard, Mudaison (FR); Olivier Grard, Mudaison (FR)

(21) Appl. No.: 12/807,457

(22) Filed: Sep. 3, 2010

Related U.S. Application Data
(60) Provisional application No. 61/276,488, filed on Sep. 11, 2009.

(51) Int. Cl. A01H 5/00

(52) U.S. Cl. PLT/161

(57) ABSTRACT

‘Gradiyel’ is a new and distinct apple tree notable for its semi-upright plant habit, freely flowering and fruiting habit, mid-season flowering time, and high storage capacity.

### CROSS REFERENCE TO RELATED APPLICATIONS

This application claims priority of co-pending U.S. Provisional Application Ser. No. 61/276,488 filed Sep. 11, 2009.

### STATEMENT REGARDING FEDERALLY SPONSORED RESEARCH OR DEVELOPMENT

None

### LATIN NAME OF THE GENUS AND SPECIES OF THE PLANT CLAIMED

Malus domestica (Borkh.)

### VARIETY DENOMINATION

‘Gradiyel’

### BACKGROUND OF THE INVENTION

The present invention relates to a new and distinct cultivar of apple tree, botanically known as Malus domestica, and hereinafter referred to by the name ‘Gradiyel’.

The new apple variety ‘Gradiyel’ originated from a controlled cross in Hérault, France of Malus domestica ‘Christmas Rose’, (not patented), as the female, or seed parent, with Malus domestica, ‘Gradigold’, (not patented) as the male or pollen, parent The new Apple tree was discovered and selected by the inventors as a single plant from within the progeny of the stated cross-pollination in a controlled environment in Hérault, France in 1991.

Asexual reproduction of the new apple tree by grafting, since 1994 in Hérault, France, has demonstrated that the new variety reproduces true to type with all of the characteristics, as herein described, firmly fixed and retained through successive generations.

### SUMMARY OF THE INVENTION

The following characteristics of the new variety have been repeatedly observed and can be used to distinguish ‘Gradiyel’ as a new and distinct cultivar of apple:
1. Semi-upright plant habit.
2. Freely flowering and fruiting habit.
3. Mid-season flowering time.
4. High storage capacity.

### BRIEF DESCRIPTION OF THE PHOTOGRAPHS

FIG. 1 shows the tree of the new variety with fruit;

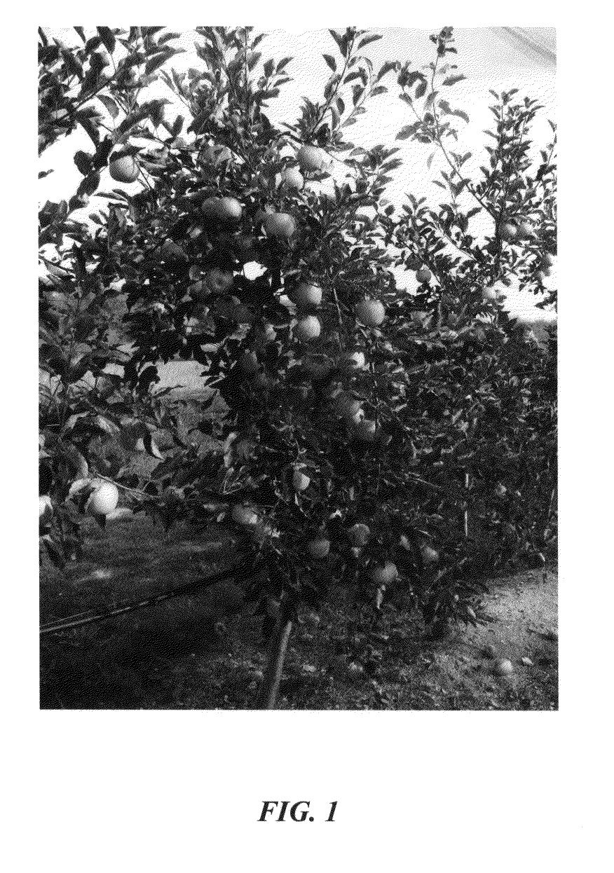

FIG. 2 shows the branch and fruit of the new variety; and

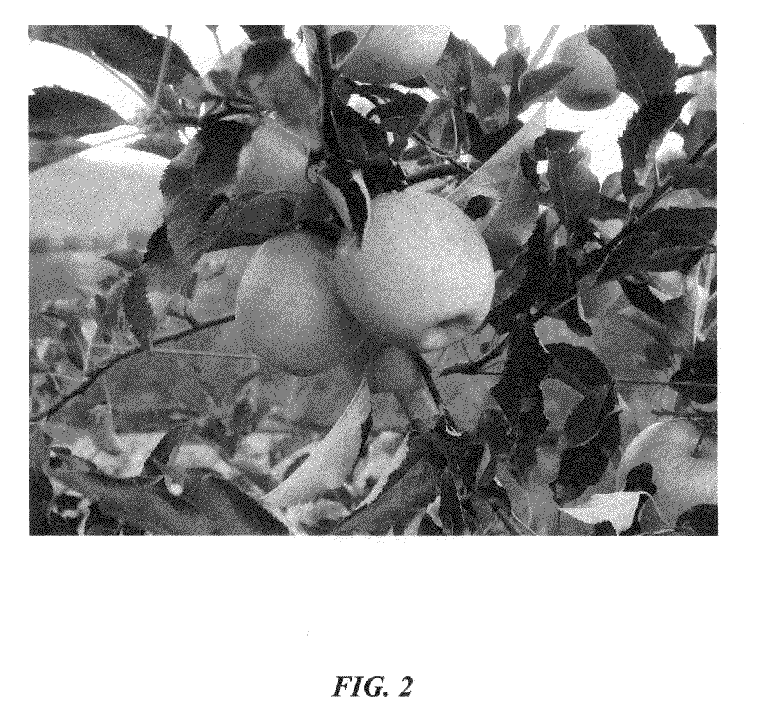

FIG. 3 shows the fruit of the new variety;

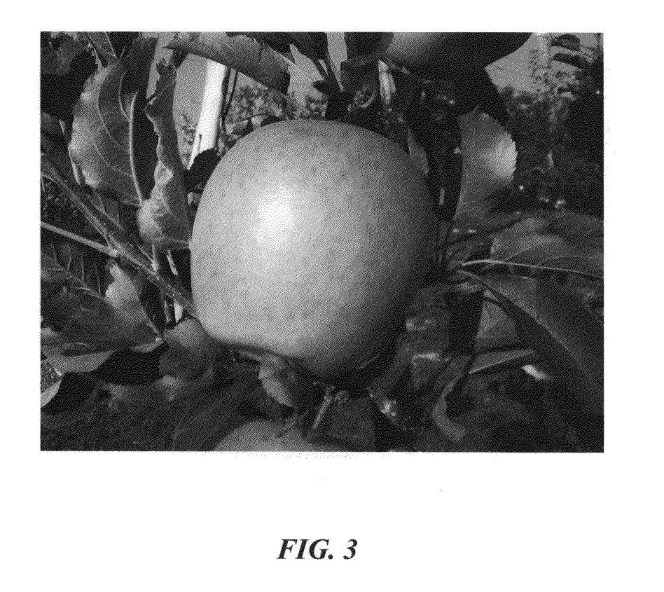

### DETAILED BOTANICAL DESCRIPTION

The following detailed botanical description is based on observations of trees planted in 1995, and described during the 2003 and 2004 growing seasons at Hérault, France. Color descriptions refer to The Royal Horticultural Society Colour Chart (1995). It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.
Botanical classification: Malus domestica Borkh cultivar Gradiyel.
Parentage:
   Female, or seed parent.—Malus domestica ‘Christmas Rose’, not patented.
   Male, or pollen, parent.—Malus domestica ‘Gradigold’ not patented.
Tree:
   Vigor.—Vigorous.
   Habit.—Semi-Upright.
   Size.—Height: 3.0 m.
   Trunk.—Diameter: 12-18 cm at 30 cm above graft union; bark texture smooth; color brown RHS 200C.
   Branches.—1 m above graft union; length 180 cm; diameter 5-7 cm; crotch angle 70°; color light brown RHS 200D.
Dormant one year old shoot:
   Size.—Diameter 5-6 mm; Length 30-40 cm.
   Color.—Brown RHS 200C.
   Internode length.—About 3 cm.
   Pubescence.—Weak.
   Lenticels.—Size 1-2 mm, density 1-2/cm².
Flowers:
   Bud.—Quantity per spur 34; length 9-11 m; shape slightly pointed; color red-purple RHS 63A.
   Petals.—Shape round; Quantity per flower 5; Margins free to touching; Length 19-20 mm; Width 13-14 mm Apex shape slightly pointed; Base shape round; upper surface color white pink RHS 65D; Lower surface color white RHS 65C when fully open.
   Flower.—Diameter of fully open flower 2-2.5 cm; Quantity per cluster 4.
   Sepals.—Quantity per flower 5; Shape triangular; color green RHS 135B.
   Pedicel.—Length 18-20 mm; Diameter 1-2 mm; Color greyed-purple RHS 187A.
   Pistil.—Length 12-13 mm; Color yellow-green RHS 145D.
   Anthers.—Quantity per flower 15-16; length 2 mm; pollen color yellow RHS 12C.
   Stigma.—Size 1 mm; Color RHS 11D.
   Style.—Length 5-6 mm; color yellow-Green RHS 145D.
   Bloom period.—Early to Mid Season; first bloom April 10, full bloom April 15 at Hérault, France.
Leaf:
   Attitude in relation to shoot.—Upwards.
   Size.—Length 10-11 cm; Width 2-3 cm; Length to Width Ratio 5-3.7.
   Margin.—Serrate.
   Color.—Upper surface yellow-green RHS 147B; lower surface yellow-green RHS 147C.
   Shape.—Ovate; apex acuminate; base aequilateral.
   Petiole.—Length 3-4 cm; width 0.5-1 mm; color yellow-green RHS 147D.
Fruit:
   Size.—Weight 150 g; diameter 75-80 mm.
   Fruit shape.—Cylindrical.
   Position of maximum diameter.—Top.
   Ribbing.—Absent.
   Aperture of eye.—Closed.
   Depth of eye basin.—8-10 mm.
   Width of eye basin.—35-40 mm.
   Stalk.—Diameter 0.5-1 mm; length 25-28 mm; Color RHS 199C.
   Depth of stalk cavity.—13-15 mm.
   Width of stalk cavity.—35 mm.
   Lenticels.—Size 0.6-0.8 mm; density 7-9/cm².
   Bloom of skin.—Absent.
   Greasiness of skin.—Absent.
   Ground color of skin.—Yellow-green.
   Over color of skin.—Pink-red.
   Amount of over color.—40-70 percent.
   Pattern of over color.—Solid flush with weakly defined stripes.
   Flesh.—Texture juicy, firm; yellow-white RHS 158D.
   Brix.—14-16.
   Seeds.—Quantity per fruit 6-8; teardrop shape; color brown RHS 200B.
   Aroma.—Strong.
   Harvest date.—Late: October 5.
   Resistance/susceptibility to known diseases/pests.—Susceptible to grey aphids.
   Keeping quality.—Good.

1. We claim a new and distinct apple tree substantially as shown and described herein.
